(19) United States
(12) Patent Application Publication
(10) Pub. No.: US 2026/0054031 A1
(43) Pub. Date: Feb. 26, 2026

(54) HEAT SHRINK TUBING-FREE CATHETER CONNECTIONS

(71) Applicant: BIOTRONIK AG, Buelach (CH)

(72) Inventors: Matthias Wesselmann, Ruedlingen (CH); Christoph Hannes Hoser, Zuerich (CH); Begije Dacic, Rorbas (CH); Morten Klabunde, Winterthur (CH); Bodo Quint, Dettighofen (DE); Nadine Troes, Buelach (CH); Christian Vivori, Duebendorf (CH)

(21) Appl. No.: 19/104,751

(22) PCT Filed: Aug. 24, 2023
(86) PCT No.: PCT/EP2023/073247

§ 371 Date: Feb. 19, 2025

(30) Foreign Application Priority Data
Aug. 30, 2022 (EP) 22192874.0

(51) Int. Cl. A61M 25/00

(52) U.S. Cl. CPC A61M 25/0009; A61M 2207/10

(57) ABSTRACT

A method for joining at least a first and a second component of a catheter using a clamping tool and heat treatment, without the use of heat shrink tubing. A device for joining at least two catheter components of a catheter includes or consists of: a clamping tool, a heating device, and optionally a cooling device. A (heat shrink tubing-free) catheter or catheter part, preferably a guide wire exit port, can be obtained by joining of at least two catheter components of the catheter by using the device or and/or the method.

### PRIORITY CLAIM

This application is a 35 U.S.C. 371 US National Phase and claims priority under 35 U.S.C. § 119, 35 U.S.C. 365(b) and all applicable statutes and treaties from prior PCT Application PCT/EP2023/073247, which was filed Aug. 24, 2023, which application claimed priority from EP Application 22192874.0, which was filed Aug. 30, 2022.

### FIELD OF THE INVENTION

A field of the invention concerns catheters, and methods to assemble catheters.

### BACKGROUND

A heat shrink tubing is usually used to create a joining pressure when generating a melt connection between a first tubular component and a second tubular component of a catheter. The heat shrink tubing is usually heated from the outside, so that it begins to shrink and presses the components to be joined together. Energy continues to be added until the encased thermoplastics melt and thus the components weld together. The shrink tubing is not reusable and can only be removed by hand which can be a cumbersome process.

Such processes have disadvantages in that the heat shrink tubing can be used only once, is relatively expensive, difficult to apply, and has to be peeled off manually. There are numerous other disadvantages. The heat shrink tubing can apply pressure only to the extremities of asymmetrical parts to be joined. There may be zones that do not experience joining pressure. The wall thickness is reduced at the extremities, which reduces the joining pressure. The heat shrink tubing cannot guarantee uniform wall thickness after joining/welding in case of non-rotationally symmetrical parts. Apart from being cumbersome, removal of the heat shrink tubing is not reliable, and bears the risk of damaging the joined components, e.g. in case the heat shrink tubing cannot be torn along the axial direction of the catheter, but cracks transversally.

### SUMMARY OF THE INVENTION

A method for joining at least two catheter components of a catheter according to an embodiment includes providing a clamping tool including a housing, at least one clamping element and an elastic component. The at least one clamping element and the elastic component are configured to be arranged within the housing. The at least one clamping element includes a clamping element opening or recess configured to arrange the at least two catheter components therein. The elastic component includes an elastic component opening or recess configured to arrange the at least two catheter components therein. The elastic component includes ferromagnetic particles,

[symbol]

At least one electrical and/or thermal conductor are arranged in an internal space or in a lumen of at least one of the at least two catheter components.

The at least two catheter components are arranged within the clamping element opening or recess and within the elastic component opening or recess.

The at least one clamping element is pressed against the elastic component thereby applying a joining pressure to at least one of the at least two catheter components.

Subsequently or simultaneously The at least two catheter components are heated while applying the joining pressure to at least one of the at least two catheter components to obtain joined components.

The joined components are then removed from the clamping tool.

### BRIEF DESCRIPTION OF THE DRAWINGS

In the following embodiments of the present invention as well as further features and embodiments of the present invention shall be described with reference to the Figures, wherein

FIG. 1A-E show a schematic illustration of steps of an embodiment of a method according to the present invention,

FIG. 2A/B show an example of a catheter made by a method of the present invention before and after heat treatment,

FIG. 3A/B show a cross-section of a clamping tool,

FIG. 4A/B show a cross-section of a catheter formed with a method according to the present invention, the catheter including a lateral opening for a guide wire, and

FIG. 5A/B show a cross-section of the catheter section shown in FIG. 4A/B,

FIG. 6A/B show a cross-section of two catheter components being arranged in a clamping tool,

FIG. 7 shows a cross-section of a catheter with a guidewire exit port.

### DETAILED DESCRIPTION OF THE PREFERRED EMBODIMENTS

A method for joining at least one catheter component of a catheter according to an embodiment includes or consists of following the steps:
providing a clamping tool including a housing, at least one clamping element and an elastic component, wherein at least one clamping element and the elastic component being arrangeable or arranged within the housing, wherein the at least one clamping element includes a clamping element opening or recess for arranging the at least one catheter component therein and the elastic component includes an elastic component opening or recess for arranging the at least one catheter component therein,
arranging at least one electrical and/or thermal conductor in an internal space or in a lumen of the at least one catheter component, and
arranging the at least one catheter component within the clamping element opening or recess and within the elastic component opening or recess, optionally arranging the at least one clamping element and the elastic component with the at least one catheter component arranged therein within the housing,
pressing the at least one clamping element against the elastic component thereby applying a joining pressure to the at least one catheter component, and
subsequently or simultaneously heating the at least one catheter component while applying the joining pressure to the at least one catheter component to obtain a joined component, and
removing the joined component from the clamping tool, and optionally removing at least one electrical and/or thermal conductor from the joined component.

The elastic component may include ferromagnetic particles, preferably including or consisting of iron. The ferromagnetic particles can enable a better heat distribution within the elastic component and thus a more efficient joining process.

In a preferred embodiment a method for joining at least two catheter components of a catheter is described, the method includes or consists of following the steps of:
providing a clamping tool including a housing, at least one clamping element and an elastic component, wherein at least one clamping element and the elastic component being arrangeable or arranged within the housing, wherein the at least one clamping element includes a clamping element opening or recess for arranging the at least two catheter components therein and the elastic component includes an elastic component opening or recess for arranging the at least two catheter components therein,
arranging at least one electrical and/or thermal conductor in an internal space or in a lumen of at least one of the at least two catheter components, and
arranging the at least two catheter components within the clamping element opening or recess and within the elastic component opening or recess, optionally arranging the at least one clamping element and the elastic component with the at least two catheter components arranged therein within the housing,
pressing the at least one clamping element against the elastic component thereby applying a joining pressure to at least one of the at least two catheter components, and
subsequently or simultaneously heating the at least two catheter components while in 5 applying the joining pressure to at least one of the at least two catheter components to obtain joined components, and
removing the joined components from the clamping tool, and optionally removing at least one electrical and/or thermal conductor from the joined components.

In case that not all (tubular) electrical and/or thermal conductors are removed from the joined components the remaining at least one (tubular) electrical and/or thermal conductor can act as a hypotube.

According to a preferred embodiment, the method is carried out without the use of a heat shrink tubing (also called heat shrink tube or shrink hose). This is advantageous as no heat shrinking tubing needs to be assembled on the components and thus has not to be removed afterwards. Not using a heat shrinking tubing reduces the costs of joining component together and speeds up the method of joining the components. In contrast to a heat shrink tubing which usually stays within the product, the elastic component is removed after a joining the components.

In this way, the method according to the present invention allows to generate a pressure-resistant, tensile catheter joint which is also atraumatic (e.g. includes a smooth surface), wherein in contrast to using a heat shrink for applying the joining pressure, the clamping tool for applying the joining pressure can be re-used. Thus, the invention offers the particular advantage that the clamping tool can be quickly removed from the joined components, since it is not necessary anymore to peel off the heat shrink from the joined components which can be cumbersome and consequently results in longer processing times.

According to a preferred embodiment of the method, the elastic component is formed from a material that is selected from the group included of: a polymeric material, an elastomer, a silicone elastomer (i.e. a polysiloxane elastomer) or silicone rubber. The hardness has an impact on the shape of the catheter components to be joined. For obtaining circular or elliptical contours of the joined catheter components the elastic component may have a Durometer shore hardness A according to ISO 868 of more than 40, preferably from 43 to 52, more preferably from 44 to 52, most preferably from 46 to 50 or 48. For obtaining other contours of the joined catheter components the elastic component may have a Durometer shore hardness A according to ISO 868 of less than 40. Particularly, hard silicone having a Durometer shore hardness A (ISO 868) of more than 40, preferably 43, may be used as elastic component for joining round our elliptical catheter parts, whereas soft silicone (e.g. having a Durometer shore hardness A (ISO 868) of less than 40) may be used as elastic component if an adaptation to the shape of the catheter parts inside the elastic component (e.g. silicone hollow tube) is required.

The joining pressure may be at least 3 bar, preferably 3.5 bar.

Furthermore, preferably the elastic material of the elastic component is a material which does not adhere to polyamides, polyether block amides or thermoplastic polyurethanes when they are melted and solidified in contact with the elastic material during cooling. Furthermore, according to a preferred embodiment of the method, a melting point, a decomposition temperature or a glass transition temperature of the elastic component is above a melting point, a decomposition temperature or a glass transition temperature of the first and/or second component. Particularly, according to a preferred embodiment of the invention, the melting point (or decomposition temperature or glass transition temperature) of the elastic component is above 200° C.

Furthermore, according to a preferred embodiment of the method according to the present invention, the elastic component, the housing and the at least one clamping element is formed from a medical grade material. The requirement for a medical grade material is defined in EU Regulation 2017/745 (MDR) or ISO 10993.

The housing, the at least one clamping element (preferably two clamping elements) and the elastic component each may have an opening (e.g. a trough hole) or a recess for arranging the at least two catheter components therein.

The at least two catheter components may be an inner catheter shaft and an outer catheter shaft or a catheter balloon and an inner shaft and/or outer shaft or a catheter balloon and a distal catheter tip.

The elastic component may be a single piece or several pieces acting together. For example, the elastic component may be an annular or rectangular elastic component with a through hole. The elastic component may be composed of two half rings acting together as a ring, wherein each half ring has a recess. The recesses of the half rings acting together than form a trough hole. The elastic component may have a wall thickness of more than 0.5 cm, preferably more than 1 cm but less than 20 cm.

The housing and the at least one clamping element, preferably two clamping elements, may have a hardness and/or stiffness that is higher than that of the elastic component or the housing or the one clamping element, at least two clamping elements, may be more rigid than the elastic component. This enables a higher compression of the elastic component from a longitudinal and a radial direction, so that the opening (e.g. the through hole) of the elastic component is reduced in size (as the material of the elastic component is constrained in moving in longitudinal direction due to the clamping elements and radially outward due to the housing).

The housing may be made of an optically transparent material (which can facilitate the positioning of the elastic component). However, the housing may be made of non-transparent material, especially when the positioning is ensured by other means (e.g. automated positioning at a known distance/position). For example, the housing may be made of a metal (which enables a better heat conduction). Alternatively or in addition the housing may be made of a ferromagnetic material.

The housing may be a heat conducting cylinder and the at least one clamping element may be a heat conducting cylinder and/or heat conducting metal disk being arrangeable or arranged within the housing and adjacent to the elastic component. The housing may be a metal and/or ferromagnetic cylinder and the at least one clamping element may be metal and/or ferromagnetic cylinder and/or a metal and/or ferromagnetic disk being arrangeable or arranged within the housing and adjacent to the elastic component. Furthermore, according to a preferred embodiment of the method, the first and the second component may be formed from a plastic material or include such a material, respectively. Particularly, the at least two components, preferably the first and/or second component, can include or consist of a polymer, a polymer blend or a polymer mixture, preferably selected from one of: a polyamide, a polyether block amide, a thermoplastic polyurethane.

Further, according to a preferred embodiment of the method, due to the heating, the first and the second component are at least partially melted so that the joining process is a welding process, wherein the clamping tool provides the joining pressure and thus defines an outer surface of the joined components.

Furthermore, according to a preferred embodiment of the method, the first and the second component may be formed from a plastic material or include such a material, respectively.

Further, according to a preferred embodiment of the method, the first component is a tubular member of the catheter, and/or wherein the second component is another tubular member of the catheter.

Further, in a preferred embodiment of the method, the second component includes a lumen configured to receive at least one electrical and/or thermal conductor and/or a guide wire of the catheter.

Furthermore, according to a preferred embodiment of the method, the step of arranging the first and the second component in the clamping tool includes arranging the second component in a lumen of the first component, such that the first component is arranged between the elastic component and the second component.

Furthermore, according to a preferred embodiment of the method, the elastic component is an annular elastic component, so that the elastic component / clamping to tool can encompass the at least two catheter components to be joined in a circumferential direction of the catheter.

Furthermore, according to a preferred embodiment of the method, heating the at least two catheter components [for joining said components upon applying said joining pressure to the first component] includes heating said components by one of: electromagnetic induction, heat conduction, heat convection, electromagnetic radiation in the range of 690 nm to 15 cm, preferably infrared irradiation.

Heating by electromagnetic induction is also known as induction heating. Induction heating is a process in which the heating of electrically conductive materials (like metals) by electromagnetic induction takes place. An induction coil (inductor) creates an electromagnetic field within the coil which heats an electrical conductor which is positioned within the coil. Electromagnetic induction may be used for heating at least one of the at least two catheter components e.g. by inducing an electric current in an electrical conductor by a coil. Preferably, for heating the at least two catheter components by electromagnetic induction, an electrical conductor is arranged in an internal space (e.g. lumen) of the first component and preferably a further electrical conductor is arranged in a lumen of the second component, and an electric current is induced in the respective electrical conductor by an electromagnet field generated by a coil (e.g. an inductor coil). The coil may be situated on an outer side of the housing, within a wall of the housing or the coil forms a wall of the housing. The heating by electromagnetic induction may be carried out for less than 1 min, preferably less than 20 s.

Preferably, the respective conductor is removed from the catheter after joining of the components.

Furthermore, according to a preferred embodiment of the method, the respective electrical conductor is a tubular electrical conductor or includes a solid core. The tubular electrical conductor may act at the same time as a catheter hypotube shaft.

In case of heat conduction or infrared irradiation at least one thermal conductor may be arranged within an internal space or in a lumen of at least one of the at least two catheter components. The thermal conductor may be a thermally conductive tube or a thermally conductive wire (e.g. made of a metal or metal alloy).

A combination of induction and conductive heating may be preferred. This results in a higher temperature of the elastic component and thus a more efficient joining of the catheter components.

According to yet another aspect of the present invention, a device for joining at least a first and a second component of a catheter is disclosed. Particularly, the device is configured to carry out the method according to the present invention. The device includes:
a clamping tool including a housing, at least one clamping element and an elastic component, wherein at least one clamping element and the elastic component being arrangeable or arranged within the housing, wherein the at least one clamping element includes a clamping element opening or recess for arranging the at least two catheter components therein and the elastic component includes an elastic component opening or recess for arranging the at least two catheter components therein, and
a heating device, the heating device being configured to heat at least one of the at least two components for joining said components, and the heating device includes at least one electrical and/or thermal conductor configured to be arranged in an internal space or in a lumen of at least one of the at least two catheter components and
optionally a cooling device, the cooling device being configured to cool at least one of the at least two catheter components.

The elastic component may be formed from a material that is selected from a polymeric material, an elastomer, a silicone elastomer or silicone rubber and/or wherein the elastic component is an annular elastic component.

The heating device may include an electrical conductor and/or thermal conductor configured to be arranged in an internal space (e.g. lumen) of one of the at least two components. The heating device may further include a coil (e.g. an inductor) configured to induce an electrical current in the respective electrical conductor to heat the first and second component. The coil may be made of copper. The heating device may further include a frequency generator connected to the coil.

The cooling device may include a cooling gas blowing device or a cooling liquid pumping device. The cooling device is configured to pass a cooling gas (e.g. air or inert gas) or a cooling liquid (e.g.) having a temperature of less than 40° C., preferably between 20° C. and 25° C., through an integral space and/or a lumen of at least one of the at least two components.

The at least one electrical conductor may be a tubular electrical conductor and/or an electrically conductive wire (the electrical conductor may have a solid core (i.e. is not hollow)). Preferably, the conductor is an inductively heatable conductor, preferably made of a metal or metal alloy. Preferably, the inductively heatable conductor includes a lumen for passing a cooling fluid (e.g. air, inert gas or cooling liquid like water) through.

Furthermore, according to a preferred embodiment of the device, the elastic component is formed out of a material that is selected from the group included of: a polymeric material, an elastomer, a silicone elastomer (i.e. a polysiloxane elastomer) or silicone rubber.

Further, in a preferred embodiment of the device, the latter includes an actuator configured to move the at least one clamping elements (back and forth) between a first position and a second position, wherein preferably the actuator includes a pneumatic cylinder.

Furthermore, in yet another preferred embodiment of the device according to the present invention, a melting point of the elastic component is above a melting point of the first and/or second component. Particularly, according to a preferred embodiment of the device, the melting point (or decomposition point) of the elastic component is above 200° C.

Above, preferred embodiments of the method and device according to the present invention are described. The respective feature/embodiment described in conjunction with the method according to the present invention can also be used in the context of the device according to the present invention and vice versa.

Thus, a (heat shrink tubing-free) catheter or catheter part is disclosed, which has been obtained by joining at least two catheter components of the catheter by using a device or a method described herein. For example, a (heat shrink tubing-free) guidewire exit port as well as a catheter having a guidewire exit port is disclosed, which has been obtained by joining at least two catheter components of the catheter by using a device or a method described herein.

The method may be a method for joining an outer catheter shaft and an inner catheter shaft wherein the outer catheter shaft includes an outer catheter shaft lumen and a lateral opening, and wherein the inner catheter shaft is partially arranged within the outer catheter shaft lumen and protrudes through a lateral opening to an outer side of the outer catheter shaft.

A preferred embodiment is a method for making a catheter with a guidewire exit port is described, the method includes or consists of following the steps of:
providing a clamping tool including a housing, at least one clamping element and an elastic component, wherein at least one clamping element and the elastic component being arrangeable or arranged within the housing, wherein the at least one clamping element includes a clamping element opening or recess for arranging an outer catheter shaft and an inner catheter shaft therein and the elastic component includes an elastic component opening or recess for arranging the at least two catheter components therein,
providing an outer catheter shaft having an outer catheter shaft lumen, a distal opening, a proximal opening and a lateral opening, wherein an inner catheter shaft is partially arranged within the outer catheter shaft lumen and protrudes through the lateral opening to an outer side of the outer catheter shaft,
arranging at least one electrical and/or thermal conductor in the outer catheter shaft lumen, and arranging at least one electrical and/or thermal conductor in an inner catheter shaft lumen,
arranging the outer catheter shaft and the inner catheter shaft with the electrical and/or thermal conductors arranged therein within the clamping element opening or recess and within the elastic component opening or recess, optionally arranging the at least one clamping element and the elastic component with the outer catheter shaft and the inner catheter shaft with the electrical and/or thermal conductors arranged therein within the housing,
pressing the at least one clamping element against the elastic component thereby applying a joining pressure the outer catheter shaft and to at least a part of the inner shaft, and
subsequently or simultaneously heating the outer shaft and the inner shaft while applying the joining pressure to the outer shaft and the inner shaft to obtain the catheter with a guidewire exit port, and
removing the catheter with the guidewire exit port from the clamping tool, and optionally removing at least one electrical and/or thermal conductor from the joined components.

Instead of providing the outer catheter shaft having the outer shaft lumen, the distal opening, the proximal opening and the lateral opening, and having the inner catheter shaft partially arranged within the outer shaft lumen and protruding through the lateral opening to the outer side of the outer catheter shaft, an outer catheter shaft having an outer catheter shaft lumen, a distal opening and a proximal opening can be provided. A lateral opening of the outer catheter shaft may then be created, e.g. by piercing an outer catheter shaft wall with a piercing element (like a needle). Then an inner catheter shaft is arranged in a way that it is partially arranged within the outer shaft lumen and protrudes through the lateral opening to the outer side of the outer catheter shaft.

In case that not all (tubular) electrical and/or thermal conductors are removed from the catheter with the guidewire exit port the remaining at least one (tubular) electrical and/or thermal conductor can act as a hypotube.

The specific features of the method for joining at least two catheter components mentioned above can also apply to the method for making a catheter with a guidewire exit port. For example, in the method for making a catheter with a guidewire exit port the elastic component may include ferromagnetic particles, preferably including or consisting of iron.

### EXAMPLES

Different Tests for joining two catheter components were carried out. In the examples 1 to 4 an induction heating system (H. Sigrist and Partner AG) with 2.8 kW was used for induction heating. The power was set to 95%. Two catheter components were inserted in a hollow cylinder shaped elastic component (e.g. made of a silicon preferably including ferromagnetic particles). The elastic component was inserted in a tubular housing and both were surrounded by a copper coil for creating an electromagnetic field. Optionally a thermal conductor (a wire, a disc washer or a disk) was added. The disc washer and/or the disc can function as a clamping tool. The wire, the disc washer and the disk may be made of metallic and/or ferromagnetic material.

### Example 1

Two polymeric catheter components were joined by induction heating only. A silicone cylinder with iron particles (30 wt %) was used as an elastic component and for magnetic induction heating. After an induction time of 25 s the temperature within the silicone was 35° C.

### Example 2

Two polymeric catheter components were joined by induction heating and additional heating (conductive heating, infrared-heating). In addition to the elastic component of example 1 which was used for magnetic induction additional heating of a washer disc which was in contact with the elastic component was used to transfer external heat to the elastic component. After an induction time of 25 s the temperature within the silicone was 45° C.

A washer disk is a hollow metal cylinder (metal cylinder having a through hole).

### Example 3

Two polymeric catheter components were joined by induction heating and additional heating (conductive heating, infrared-heating). In addition to the elastic component of example 1 which was used for magnetic induction additional heating of a metal disc which was in contact with the elastic component was used to transfer external heat to the elastic component. After an induction time of 4 s the temperature within the silicone was 85° C.

A disk is a metal cylinder (having no through hole).

### Example 4

Two polymeric catheter components were joined by induction heating and additional heating (conductive heating, infrared-heating). In addition to the elastic component of example 1 which was used for magnetic induction heating a metal wire which was inserted into the catheter components was used to transfer external heat to the elastic component. After an induction time of 4 s the temperature within the silicone was 100° C.

As can be seen from Examples 1 to 4 a combination of induction heating and external heating results in a higher temperature of the elastic component. Using a wire inserted into the elastic component cylinder achieved the highest temperature in a very short time. Depending on the polymer(s) of the catheter components to be joined a combination of induction heating and conductive heating might be preferred.

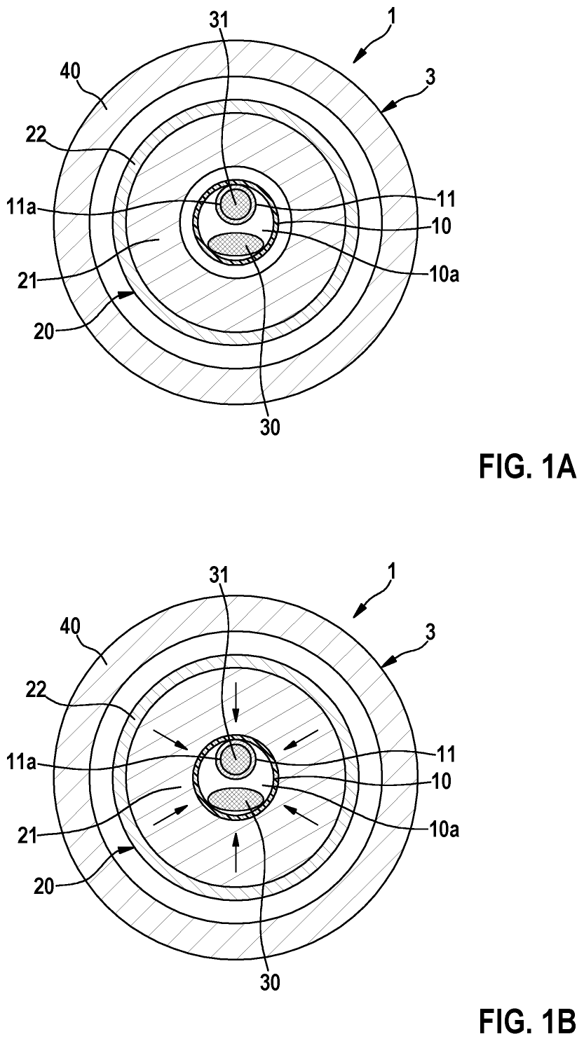

The present invention utilizes a clamping tool 20 as shown in FIG. 1. The clamping tool 20 includes an elastic component 21, e.g. in the form of a rubber-like mold covering the area to be joined. Particularly, the elastic component 21 can be an annular member surrounding an opening for receiving components 10, 11 to be joined to one another. Particularly, pressure applied to the elastic component 21 via the clamping elements 23 constricts the opening until it is supported against the surface of the molded components, here an outer surface of the first component 10. Particularly, the energy for heating and melting said components 10, 11 to be joined can be introduced by conventional methods, such as induction, heat conduction, convection, radiation. According to a preferred embodiment, electromagnetic induction is used for heating the components 10, 11 by inducing electric currents in electrical conductors 30, 31 (Joule heating) by a coil 40 and a frequency generator connected thereto as will be described further below.

Furthermore, preferably, the clamping tool 20 includes an actuator, that is configured to compress the elastic component 21. Particularly, said elastic component can only deflect inwards and thus presses on the components 10, 11 inserted into the opening delimited by the elastic component 21.

FIGS. 1A to 1E shows cross-sectional views of a joining process of a first and a second component 10, 11 of a catheter using the above-described clamping tool 20. FIG. 1A to 1E also show a device 1 for joining at least two catheter components 10, 11 of a catheter including a clamping tool 20 and a heating device 3. The clamping tool 20 includes a housing 22, an elastic component 21 and at least one clamping element (not shown in this cross-sectional view, as the cross-section runs through the elastic component 21),

In a first step shown in FIG. 1A, the first and the second component 10, 11 are arranged in the clamping tool 20 such that the elastic component 21 encompasses the first component 10, wherein the second component 11 is arranged in an internal space/lumen 10a of the first component. Here, both components are elongated tubular components of the catheter. Furthermore, in order to able to heat the first and second component 10, 11 by way of electromagnetic induction, an electrical conductor 30 is arranged in the lumen of the first component 10 and a further electrical conductor 31 is arranged in the lumen 11a of the second component 11.

Then, as indicated in FIG. 1B a pressure is exerted on the elastic component 21 which is thereby moved from a non-compressed state (FIG. 1A) into a compressed state where the elastic component presses the outer first component 10 towards the inner second component 11 to provide a joining pressure to the components 10, 11 to be joined.

Furthermore, in order to join said components 10, 11, an electromagnetic current is induced in the conductors 10, 11 to heat the conductors 10, 11 and thereby melt the adjacent first and second component 10, 11 (cf. FIG. 1C).

Due to the joining pressure provided by the elastic component 21, the latter presses the molting first component towards the surfaces provided by the second component and the conductor 30 arranged in the lumen 10a of the first component so that the first component is joined with the second component therein to form joined components 12. The joined components 12 includes two lumens 10a, 11a defined by the cross-sectional contour of the electrical conductors 30, 31(cf. FIG. 1D).

Eventually, the elastic component is expanded (e.g. by means releasing the pressure) so that the clamping tool 20 again assumes its second state and releases the joined components 12 of catheter (cf. FIG. 1E).

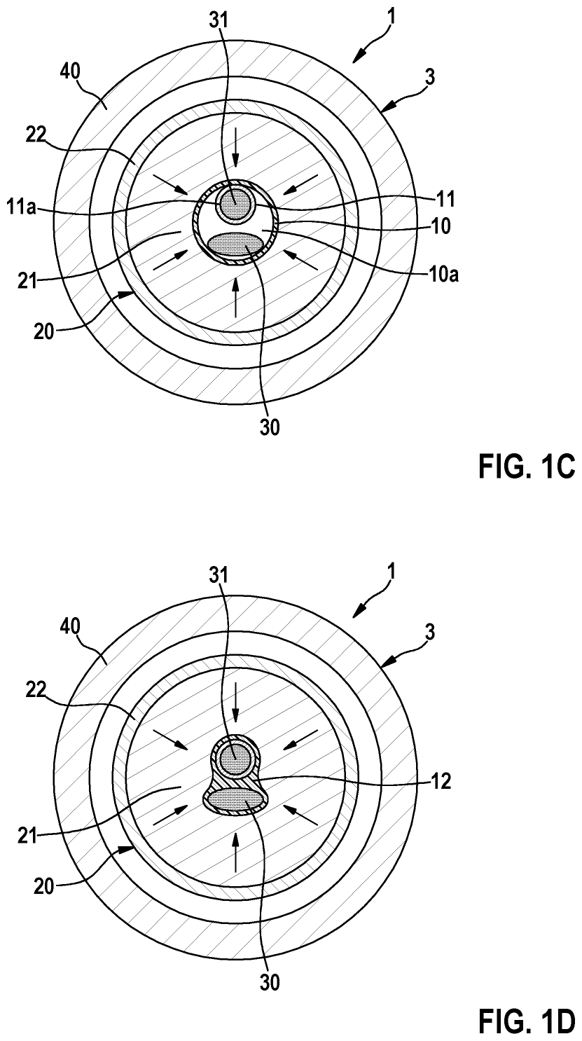

Furthermore, FIGS. 2A and 2B show an example of the present invention, wherein here two ferromagnetic wires 30 are pushed into a tube 10 made of a polymer, e.g. a polyamide like PA12. The assembly is inserted into the clamping tool described in conjunction with FIG. 1.

For welding, the clamping tool 20 is closed with 5 bar and the tube 10 is thus already pressed against the contour of the conductors 30 as far as the bending moment of the tube 10 allows. The wires 30 are heated by induction for 1 s to 8 s, preferably 6 s, so that the thermoplastic material of the tube 10 begins to flow. After a short cooling period of (approx. 2 s), the clamping tool 20 is released and the formed part can be removed. The left-hand side of FIG. 2 shows the cross section of the experimental setup with the polyamide (PA) tube 10 and the wires 30. The right-hand side of FIG. 2 shows the result after welding.

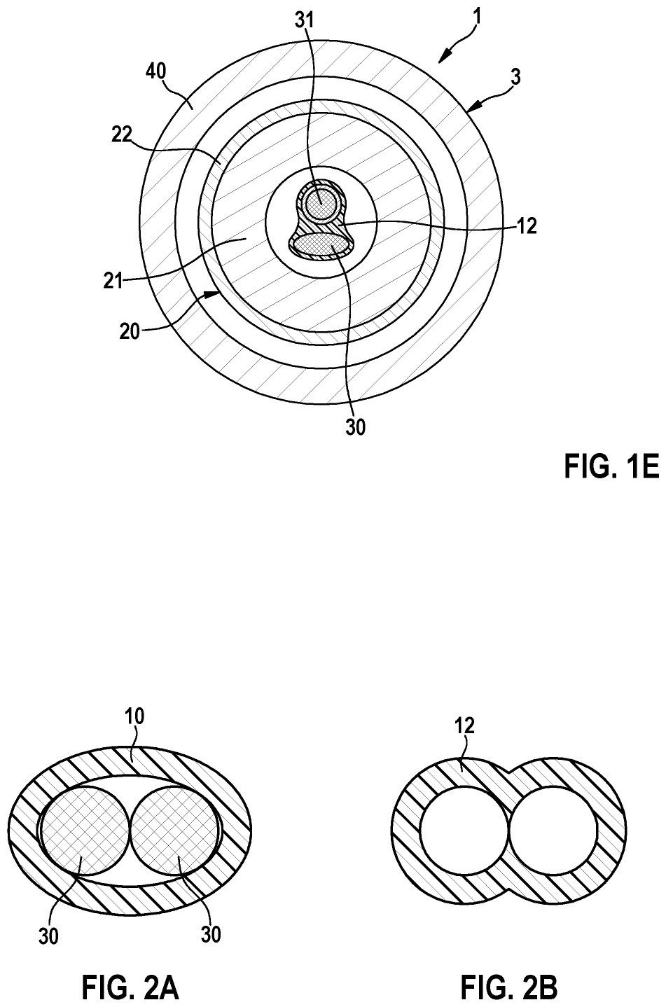

FIGS. 3A and 3B show a cross-sectional view of a clamping tool 20 being arranged within a coil 40. The clamping tool 20 including a housing 22, an elastic component 21 and two clamping elements arranged within the housing. The elastic component 21 and the two clamping elements have a through hole. The housing 22 has two openings. FIG. 3A shows the clamping tool in a first state, wherein the elastic component 21 cannot contact the catheter components (not shown). FIG. 3B shows the clamping tool 20 in a second state, wherein the elastic component 21 can contact at least one of the at least two catheter components (not shown) and to provide a joining pressure to at least one of the at least two components.

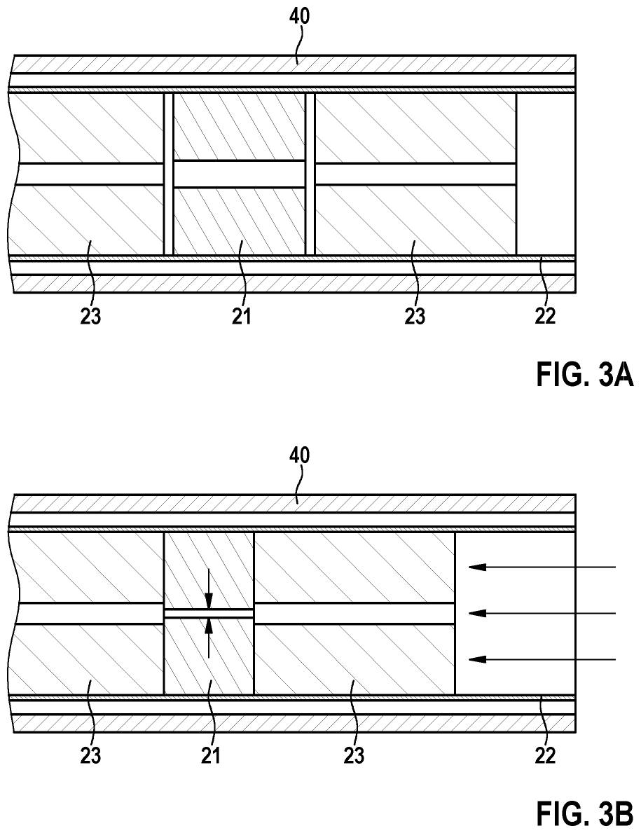

FIGS. 4A and 4B show a further example of the present invention, wherein here catheter with a guide wire exit port 5 is generated. A first component 10 in form of an outer shaft (of a distal anterior part for the construction of a monorail catheter) having an internal space/inner lumen 10a is pierced, e.g. with a needle, to form a hole for a guide wire exit port. A second component 11 in form of an inner shaft having an inner lumen 11a is inserted into the hole and aligned to match the outer shaft 10 (in the case of balloon catheters, for example, by symmetrically aligning the X-ray markers to the balloon, which is already connected to the outer shaft 10). An inductively heatable conductor 31 is inserted into the inner shaft, which will later heat inner shaft in the area of the weld. Furthermore, another electrical conductor 30 is inserted adjacent to the inner shaft into the outer shaft 10. FIG. 4A shows the catheter with the individual components 10, 11 whereas FIG. 4B shows the catheter with the joined components 12. The outer shaft with inner shaft and electrical conductors 31, 30 is now inserted into the clamping tool 20 and induction coil 40 (cf. FIG. 1). The conductors 31, 30 may have a biocompatible non-stick coating (e.g. silicone oil and others) to make it easier to remove them after welding.

In case of manufacturing a guidewire exit port the electrical conductor 31 can be a tubular electrical conductor which can be left in the catheter to act as a hypotube.

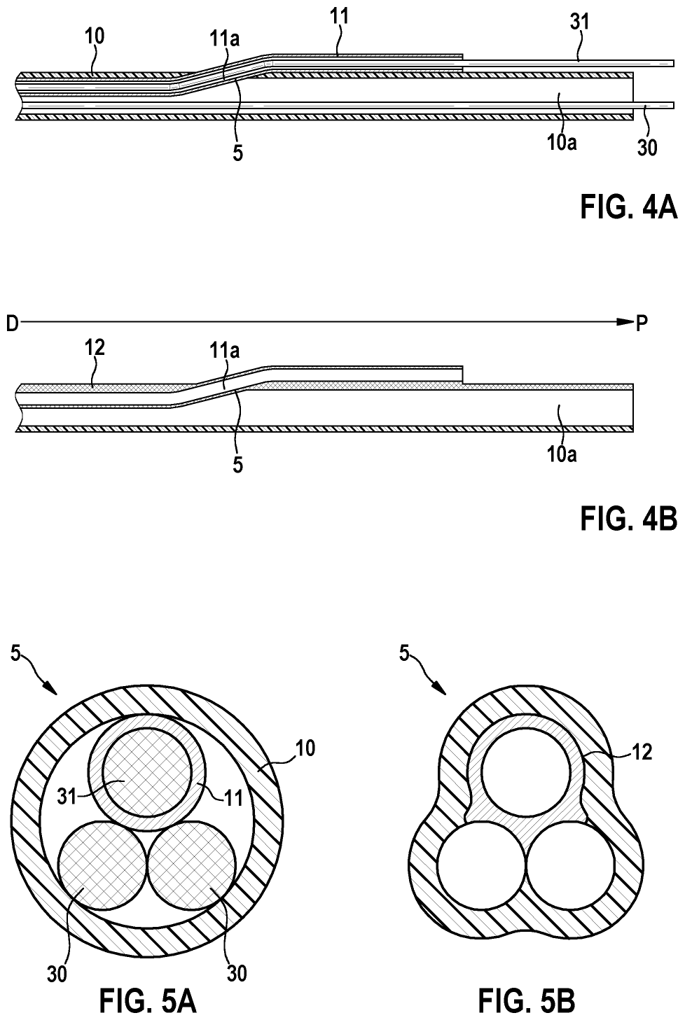

FIG. 5A shows a cross section of the setup with a first component 10, e.g. a polymer tube, preferably a polyamide (PA) tube, and the conductors 30, 31 and the second component (e.g. inner shaft) 11 therein. The lower two conductors 30 substitute for core welding profile (to keep inflation lumen open) which also heats up and introduces heat from the inside. FIG. 5B shows the result after welding, while pressure is applied by the clamping tool 20. FIG. 5A shows the catheter with the individual components 10, 11 whereas FIG. 5B shows the catheter with the joined components 12.

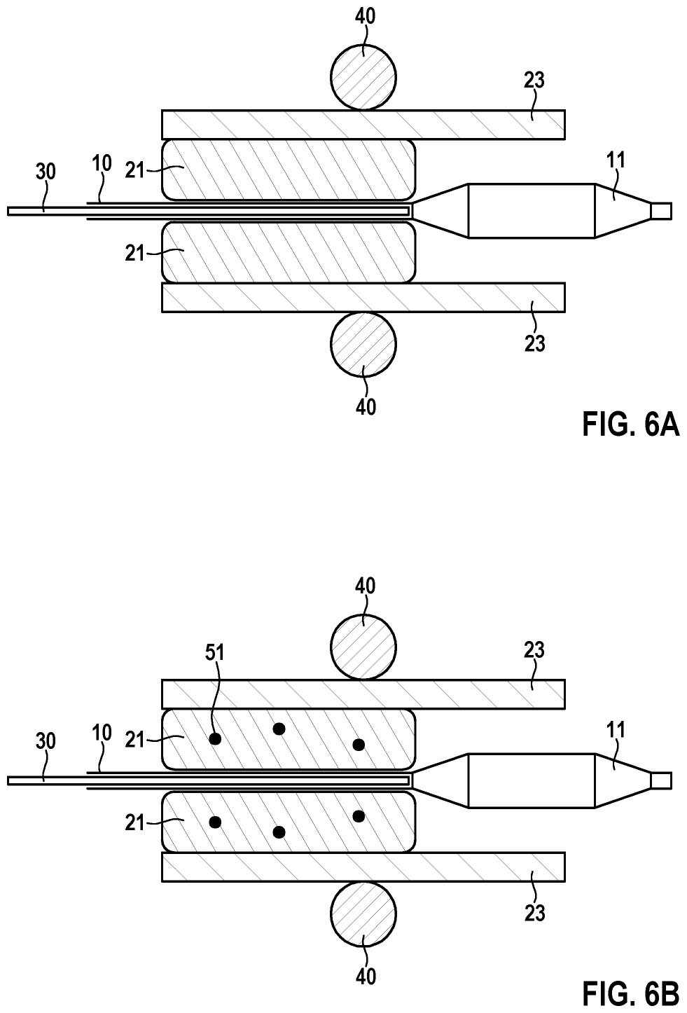

FIG. 6A and FIG. 6B show a cross-section of at least two catheter components 10, 11 (here catheter shaft 10 and balloon catheter11) being surrounded by an elastic component. In FIG. 6A the elastic component includes no ferromagnetic particles whereas in FIG. 6B the elastic component includes ferromagnetic particles. The elastic component is arranged within a housing 22 or clamping element which is surrounded by a copper coil 40. The elastic component 21 has a hollow cylindrical shape. The housing 22 has two openings. An electrical conductor is inserted in the catheter shaft. The balloon neck and the catheter shaft are overlapping each other partially, so that they can be thermally joined.

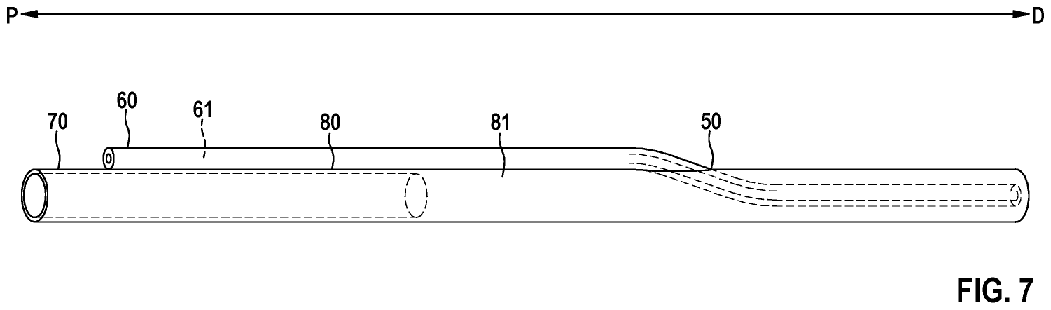

FIG. 7 shows a cross-section of a catheter with a guidewire exit port 50 and at least two catheter components, namely a first component 80 and second component 60, that was thermally joined by a method according to the invention. The first component 80 (here in the in form of an outer catheter shaft) has an inner lumen 81 and a lateral opening (e.g. a hole or slit). The first component 80 further has a distal (D) opening and a proximal (P) opening. The second component 60 (here in the form of an inner catheter shaft) has an inner lumen 60 for a guidewire. The second component 60 is inserted into the lateral opening of the outer catheter shaft. The second component 60 is partially arranged within the inner lumen 81 of the first component 80 and partially arranged outside the first component, preferably on an outer side of the first component. A metallic hypotube shaft 70 is partially arranged within the inner lumen 81 of the first component (not being in contact with the first component and not being parallel or coaxially arranged to the first component). The metallic hypotube shaft 70 is situated in a part of the first component 80 being arranged proximal to the guidewire exit port 50. However, the metallic hypotube shaft may also be omitted.

The present invention offers the advantage that the consumption of heat shrink tubing can be avoided. Furthermore, the method is easier to automate, since the components can be easily inserted into the clamping tool and removed therefrom. Particularly, no time is spent for attaching and removing the heat shrink tubing. Furthermore, a uniform and very high joining pressure is possible, wherein the joining pressure is adjustable via pressure feed and material of the elastic component. Furthermore, a uniform wall thickness/material distribution even after welding can be achieved.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. A method for joining at least two catheter components of a catheter, comprising:
providing a clamping tool comprising a housing, at least one clamping element and an elastic component, wherein the at least one clamping element and the elastic component are configured to be arranged within the housing, wherein the at least one clamping element comprises a clamping element opening or recess configured to arrange the at least two catheter components therein and the elastic component comprises an elastic component opening or recess configured to arrange the at least two catheter components therein, and wherein the elastic component comprises ferromagnetic particles,
arranging at least one electrical and/or thermal conductor in an internal space or in a lumen of at least one of the at least two catheter components,
arranging the at least two catheter components within the clamping element opening or recess and within the elastic component opening or recess,
pressing the at least one clamping element against the elastic component thereby applying a joining pressure to at least one of the at least two catheter components,
subsequently or simultaneously heating the at least two catheter components while applying the joining pressure to at least one of the at least two catheter components to obtain joined components, and
removing the joined components from the clamping tool.

2. The method according to claim 1, wherein the elastic component is formed from a material that is selected from a polymeric material, an elastomer, a silicone elastomer or silicone rubber and/ or wherein the elastic component is an annular elastic component.

3. The method according to claim 2, wherein a melting point, a decomposition temperature or a glass transition temperature of the material of the elastic component is above a melting point, a decomposition temperature or a glass transition temperature of each of the at least two catheter components.

4. The method according to claim 1, wherein each of the at least two catheter components comprises a polymer, a polymer blend, polymer composite or a polymer mixture.

5. (canceled)

6. The method according to claim 1, wherein the at least two catheter components comprise a first component and a second component, wherein the first component is a tubular member of the catheter, and/or wherein the second component is a tubular member of the catheter.

7. The method according to claim 6, wherein the step of arranging the first and the second component in the clamping tool comprises arranging the second component in an internal space of the first component such that the first component is arranged between the elastic component and the second component.

8. (canceled)

9. The method according to claim 1, wherein the heating comprises heating by electromagnetic induction, heat conduction and/or electromagnetic radiation with a wavelength in the range of 690 nm to 15 cm.

10-15. (canceled)

16. The method according to wherein the ferromagnetic particles comprise iron.

17. The method according to claim 1, wherein the housing is a heat conducting cylinder and the at least one clamping element is heat conducting cylinder and/or heat conducting metal disk configured to be arranged within the housing and adjacent to the elastic component.

18. The method according to claim 1, wherein the housing is a metal and/or ferromagnetic cylinder and the at least one clamping element is a metal and/or ferromagnetic cylinder and/or a metal and/or ferromagnetic disk configured to be arranged within the housing and adjacent to the elastic component.

19-23. (canceled)

24. A device for joining at least two catheter components of a catheter, comprising:
a clamping tool comprising a housing, at least one clamping element and an elastic component, wherein at least one clamping element and the elastic component are configured to be arranged within the housing, wherein the at least one clamping element comprises a clamping element opening or recess for arranging the at least two catheter components therein and the elastic component comprises an elastic component opening or recess for arranging the at least two catheter components therein, and wherein the elastic component comprises ferromagnetic particles, and
a heating device configured to heat at least one of the at least two components to join said components, and the heating device comprises at least one electrical and/or thermal conductor configured to be arranged in an internal space or in a lumen of at least one of the at least two catheter components.

25. The device according to claim 24, wherein the heating device comprises at least one electrical conductor configured to be arranged in an internal space and/or in a lumen of at least one of the at least two catheter components, wherein the heating device comprises a coil configured to induce an electrical current in the at least one electrical conductor to heat at least one of the at least two catheter components.

26. The device according to claim 25, wherein the at least one electrical conductor is a tubular electrical conductor or an electrically conductive wire and/or wherein the coil is situated on an outer side of the housing, within a wall of the housing or forms a wall of the housing.

27. (canceled)

28. The device according to claim 24, wherein the housing is a heat conducting cylinder and the at least one clamping element is heat conducting cylinder and/or heat conducting metal disk configured to be arranged within the housing and adjacent to the elastic component.

29. The device according to claim 1, wherein the housing is a metal and/or ferromagnetic cylinder and the at least one clamping element is metal and/or ferromagnetic cylinder and/or a metal and/or ferromagnetic disk being arrangeable or arranged within the housing and adjacent to the elastic component.

30-33. (canceled)

34. The method according to claim 1, comprising:
arranging the at least one clamping element and the elastic component with the at least two catheter components arranged therein within the housing prior to the pressing, and
removing at least one electrical and/or thermal conductor from the joined components.

35. The device according to claim 24, comprising a cooling device configured to cool at least one of the at least two catheter components.
